(19) United States
(12) Patent Application Publication
(10) Pub. No.: US 2024/0400485 A1
(43) Pub. Date: Dec. 5, 2024

(54) PROCESS FOR THE HYDROFORMYLATION OF PROPYLENE

(71) Applicants: Université de Strasbourg, Strasbourg (FR); Centre National De La Recherche Scientifique, PARIS 16 (FR)

(72) Inventors: Pawel Dydio, Kehl (DE); Michel Sigrist, Mollkirch (FR)

(21) Appl. No.: 18/714,871

(22) PCT Filed: Nov. 25, 2022
(86) PCT No.: PCT/EP2022/083343

§ 371 Date: May 30, 2024

(30) Foreign Application Priority Data
Nov. 30, 2021 (EP) 21315257.2

(51) Int. Cl. C07C 45/50

(52) U.S. Cl. CPC C07C 45/505

(57) ABSTRACT

A process for the hydroformylation of propylene with carbon monoxide and hydrogen to selectively form iso-butanal with respect to n-butanal implements a hydroformylation catalyst in combination with a phosphene. The hydroformylation catalyst is or includes a palladium catalyst that comprises PdX₂, where X is a halogen element selected from iodine I and bromine Br. The phosphine is of the form P(R)(R′)(R″), where R, R′ and R″ each comprise an alkyl group or an aryl group or an alkoxy group or an amino group.

### CROSS REFERENCE TO RELATED APPLICATIONS

This application is a national phase entry under 35 U.S.C. § 371 of International Patent Application PCT/EP2022/083343, filed Nov. 25, 2022, designating the United States of America and published as International Patent Publication WO 2023/099356 A1 on Jun. 8, 2023, which claims the benefit under Article 8 of the Patent Cooperation Treaty to European Patent Application Serial No. 21315257.2, filed Nov. 30, 2021.

### TECHNICAL FIELD

The present disclosure concerns a method of hydroformylation of propylene, which selectively produces iso-butanal with respect of n-butanal.

### BACKGROUND

Hydroformylation is a synthetic route to produce aldehydes by chemical reaction of an alkene with carbon monoxide and dihydrogen in the presence of a catalyst, typically a metal complex in combination with an alkyl or aryl phosphine. In particular, the hydroformylation of propylene is one of the most important reactions in the chemical industry. The hydroformylation of propylene produces two isomers of butanal, and more particularly n-butanal and iso-butanal, with a selectivity that depends on the reaction conditions and the nature of the catalyst used. In this respect, the catalysts generally used, which comprise Rh and/or Co-based complexes, favor the formation of n-butanal over iso-butanal.

However, the chemical industry is facing an increasing demand for iso-butanal so that a reverse selectivity in favor of iso-butanal is desirable. This objective has given rise to strong developments, particularly in academic institutes. These developments have essentially been focused on the development of phosphine ligands. In particular, and as proposed in the document [1] cited at the end of the description, ligands have been tailored to modify the intrinsic selectivity of Rh-based catalysts.

By way of example, the document [2] cited at the end of the description discloses the use of a tris-Zn-porphyrin-tris-pyridylphosphine ligand in the form of a capsule allowing selective formation of iso-butanal with respect to n-butanal. However, this selectivity is not maintained when the temperature imposed during the hydroformylation of propylene is higher than 70° C., a temperature required in industrial environments.

The paper [3] cited at the end of the description proposes another type of ligand that also allows the selective formation of iso-butanal with respect to n-butanal.

Nevertheless, the solutions proposed in these documents are not satisfactory.

Indeed, the proposed ligands are both costly and complicated to implement in the industrial environment.

It is therefore an object of the present disclosure to provide a process for the hydroformylation of propylene to selectively form iso-butanal with respect to n-butanal, which is compatible with the requirements of industrial production.

### BRIEF SUMMARY

The present disclosure relates to a process for the hydroformylation of propylene with carbon monoxide and hydrogen to selectively form iso-butanal with respect to n-butanal, the process implementing a hydroformylation catalyst combined with a phosphine,
the hydroformylation catalyst comprising a palladium catalyst that comprises the element PdX₂, where X is a halogen element selected from iodine I and bromine Br;
the phosphine is of the form P(R)(R′)(R″), where R, R′ and R″ each comprise an alkyl group or an aryl group or an alkoxy group or an amino group.

According to one embodiment, the phosphine is selected from one of the following: P(Me)₃, P(Et)₃, P(n-Bu)₃, P(n-Hex)₃, P(i-Bu)₃, P(i-Pr)₃, P(Cy)₃, P(Cyp)₃, P(t-Bu)₃, P(Bn)₃, P(Cy)₂(t-Bu), P(Cy)(t-Bu)₂, P(Me)(t-Bu)₂, P(i-Pr)₂(t-Bu), P(1-Ad)₂(n-Bu), P(Ph)(t-Bu)₂.

According to one embodiment, the molar ratio of halogen element X to palladium is in the 1 to 3 range, advantageously in the 2 to 2.5 range.

According to one embodiment, the halogen element X is iodine I.

According to one embodiment, the molar ratio of phosphine P(R)(R′)(R″) to palladium is in the 1 to 4 range, advantageously in the 2 to 3 range.

According to one embodiment, the phosphine comprises the element P(Cy)₃.

According to one embodiment, the palladium catalyst employs an additional palladium element, advantageously this additional palladium element comprises Pd(OAc)₂.

According to one embodiment, the process is carried out in a non-aqueous solvent that comprises at least one of the elements selected from: anisole, toluene, 1,4-dioxane, dimethylacetamide, p-xylene, decahydronaphthalene, DMF, DMSO, α, α, α-trifluorotoluene, hexafluorobenzene, 1,2-dichlorobenzene, methyl benzoate.

According to one embodiment, the process is carried out at a temperature of between 70° C. and 200° C., advantageously in the 80° C. to 120° C. range.

### BRIEF DESCRIPTION OF THE DRAWINGS

Further features and advantages of the embodiments of the present disclosure will be apparent from the following detailed description with reference to the attached figures in which:

FIG. 1 shows the chemical reaction associated with the hydroformylation of propylene with carbon monoxide (CO) and dihydrogen (H₂) in the presence of a catalyst according to the present disclosure;

FIG. 2 is a graphical representation of the evolution of the selectivity of the hydroformylation reaction in the presence of a phosphine of the form P(Cy)₃, and a catalyst of the type PdX₂, the vertical axis representing the selectivity while the horizontal axis lists the different catalysts employed;

FIG. 3 is a graphical representation of the evolution of the catalytic activity in the presence of an alkyl phosphine of the form P(Cy)₃, and a catalyst of the type PdX₂, the vertical axis representing the catalytic activity while the horizontal axis lists the different catalysts employed;

FIG. 4 shows the evolution of the catalytic activity as a function of the L/Pd ratio (vertical axis, “L” being a phosphine ligand) and the I⁻/Pd ratio (horizontal axis); and

FIG. 5 shows the evolution of the selectivity S as a function of the L/Pd ratio (vertical axis) and the I⁻/Pd ratio (horizontal axis).

### DETAILED DESCRIPTION

The present disclosure relates to a process for the hydroformylation of propylene with carbon monoxide and hydrogen to selectively form iso-butanal (denoted (b)) with respect to n-butanal (denoted (1)). Moreover, the selectivity S of the hydroformylation reaction is defined in the context of the present disclosure as the ratio n_{b} to nₗ, where n_{b} and nₗ are the respective quantities (for example, in moles) of iso-butanal and n-butanal obtained during the hydroformylation of propylene.

The process according to the present disclosure uses, in particular, a catalyst, referred to as a hydroformylation catalyst, combined with a phosphine. More particularly, the hydroformylation catalyst comprises the element PdX₂, where X is a halogen element chosen from iodine I or bromine Br, while the phosphine is of formula P(R)(R′)(R″), where R, R′ and R″ each comprise an alkyl group or an aryl group or an alkoxy group or an amino group.

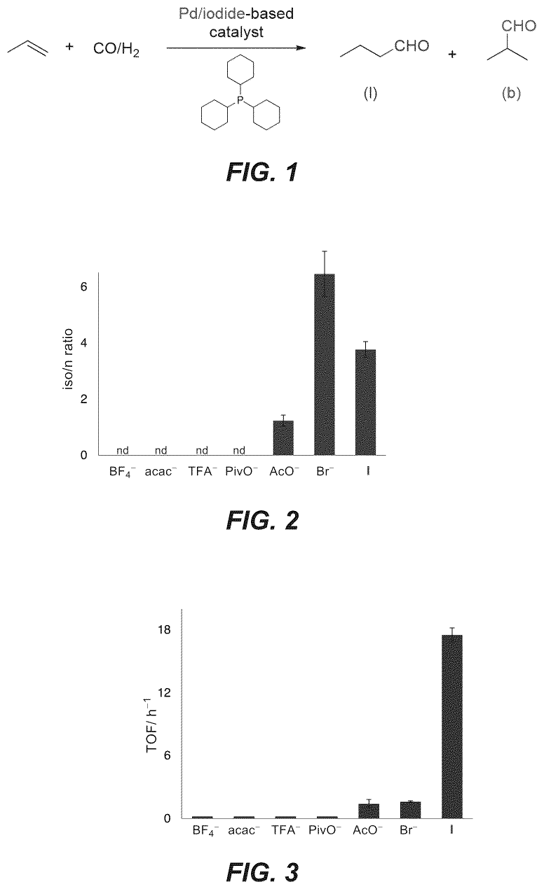

FIG. 1 shows the chemical reaction associated with the hydroformylation of propylene with carbon monoxide CO and dihydrogen in the presence of a catalyst according to the present disclosure.

It is known that such a chemical reaction leads to the formation of iso-butanal and n-butanal in proportions that depend on the reaction conditions and the nature of the catalyst and/or phosphine employed.

In this respect, in order to promote the formation of iso-butanal with respect to n-butanal, it is proposed in the present disclosure to use a palladium-based catalyst, and more particularly a catalyst that comprises PdX₂ where X is a halogen. In particular, X can comprise at least one of the elements selected from: iodine I, bromine Br.

Furthermore, and still in accordance with the terms of the present disclosure, the phosphine is of the form P(R)(R′)(R″), where R, R′ and R″ each comprise an alkyl group or an aryl group or an alkoxy group or an amino group.

FIG. 2 is a graphical representation of the evolution of the selectivity of the hydroformylation reaction of propylene in the presence of a phosphine of the form P(Cy)₃, (Cy: cyclohexyl) and a catalyst of the type PdX₂.

Notably, in this FIG. 2, the element X may comprise at least one of the anions selected from: BF₄⁻, acac⁻ (acetylacetone ion), TFA⁻ (trifluoroacetic ion), PivO⁻ (pivalic ion), AcO⁻ (acetate ion), Br⁻, I⁻.

It can be clearly observed on the graph in FIGS. 2 and 3 that catalysts bearing the ions BF₄⁻, acac⁻, TFA⁻, or PivO⁻, do not favor the formation of either iso-butanal or n-butanal. In turn, the catalyst bearing AcO⁻ favors the formation of a mixture of both products in a close to equimolar ratio. Conversely, catalysts bearing Br⁻ and I⁻ ions allow the selective formation of iso-butanal with respect to n-butanal.

More particularly, the consideration of Br⁻ or I⁻ ions increases the selectivity S above 4.

FIG. 3 illustrates the catalytic activity (TOF/h⁻¹) for each of the ions listed with reference to FIG. 2, and in the case of a phosphine of the form P(Cy)₃.

This figure also demonstrates very clearly that Br ions, as well as the I− ion, allows a catalytic activity of at least 2 h⁻¹ to be achieved and equal to 17 h⁻¹ in the case of the I⁻ ion.

The hydroformylation reaction shown in FIG. 1 is advantageously carried out at a temperature between 70° C. and 200° C., more particularly between 80° C. and 120° C.

These temperatures ensure sufficient activity of the catalyst, and more particularly reaction kinetics compatible with the requirements of industrial production.

Advantageously, the hydroformylation reaction is carried out in the presence of ligand selected from the following phosphines. P(Me)₃, P(Et)₃, P(n-Bu)₃, P(n-Hex)₃, P(i-Bu)₃, P(i-Pr)₃, P(Cy)₃, P(Cyp)₃, P(t-Bu)₃, P(Bn)₃, P(Cy)₂(t-Bu), P(Cy)(t-Bu)₂, P(Me)(t-Bu)₂, P(i-Pr)₂(tBu), P(1-Ad)₂(n-Bu), P(Ph)(t-Bu)₂.

In this respect, the following Table 1 shows the catalytic activity (TOF) as a function of the different phosphines of formula P(R)(R′)(R″) mentioned above.

TABLE 1

| Phosphine | | TOF |
|---|---|---|
| P(R)(R′)(R″) | | (iso:n ratio)^{a} |
| PCy₃ | [symbol] | 17 (4.0:1) |
| P( [symbol] )₃ | | 14 (4.4:1) |
| PCyp₃ | | 15 (3.2:1) |
| PCy₂(t-Bu) | | 19 (2.4:1) |
| [symbol] (t-Bu)₂ | | 20 (2.3:1) |
| [symbol] t-Bu)₂ | | 16 (3.9:1) |
| [symbol] ₂(t-Bu) | | 22 (2.6:1) |
| P(1-Ad)₂(n-Bu) | | 15 (2.5:1) |

The experimental conditions used to obtain these catalytic activities are as follows:
Catalyst considered: 10 μmol PdI₂;
solvent considered: 2 mL anisole;
pressure of propylene: 4 bar;
pressure of carbon monoxide: 20 bar;
pressure of dihydrogen: 80 bar;
volume of solvent: 2 mL;
imposed temperature: 100° C.

It is thus notable that each of the phosphines considered ensures sufficient activity of the catalyst, and more particularly reaction kinetics compatible with the requirements of industrial production. Phosphines cited provide an activity among of 14 h⁻¹ and 22 h⁻¹.

This table also lists the selectivity S (iso:n ratio) obtained in the presence of each of these phosphines of formula P(R)(R′)(R″).

Advantageously, R, R′ and R″ are chosen so that the selectivity S of the hydroformylation of propylene with carbon monoxide and hydrogen is above 2, advantageously above 3.

Advantageously, the hydroformylation reaction is carried out in the presence of a solvent selected from the following solvents: anisole, toluene, 1,4-dioxane, dimethylacetamide, p-xylene, decahydronaphthalene, DMF (N,N-dimethylformamide), DMSO (dimethylsulphoxide), α, α, α-trifluorotoluene, hexafluorobenzene, 1,2-dichlorobenzene, methyl benzate.

In this respect, the following Table 2 shows the catalytic activity (TOF) as a function of the different solvents mentioned above.

TABLE 2

| | TOF |
|---|---|
| Solvent | (iso:n ratio) |
| anisole | 17 (4.0:1) |
| toluene | 7 (3.8:1) |
| 1,4-dioxane | 7 (4.4:1) |
| dimethylacetamide | 7 (4.6:1) |
| p-xylene | 6 (2.9:1) |
| decahydronaphthalene | 3 (2.1:1) |
| DMF | 5 (2.4:1) |
| DMSO | 1 (5.2:1) |
| CF₃Toluene | 8 (3.4:1) |
| Hexafluorobenzene | 4 (2.9:1) |
| 1,2-dichlorobenzene | 13 (3.2:1) |
| Methyl benzoate | 9 (3.8:1) |

The experimental conditions used to obtain these catalytic activities are as follows:
Catalyst considered: 10 μmol PdI₂;
ligand considered: 20 μmol PCy₃;
pressure of propylene: 4 bar;
pressure of carbon monoxide: 20 bar;
pressure of dihydrogen: 80 bar;
volume of solvent: 2 mL;
imposed temperature: 100° C.

It is thus notable that each of the solvents considered ensures the sufficient activity of the catalyst, and more particularly reaction kinetics compatible with the requirements of industrial production.

Anisole and 1,2-dichlorobenzene provide an activity of 17 h⁻¹ and 13 h⁻¹, respectively.

This table also lists the selectivity S (iso:n ratio) obtained in the presence of each of these solvents. The latter, whatever the solvent considered, remains favorable to the formation of iso-butanal compared to n-butanal.

According to the present disclosure, it is also possible to consider different molar ratios X to Pd. In particular, the molar ratios X to Pd can be adjusted by considering a catalyst formed by a mixture of Pd(OAc)₂, PdX₂, PPh₄X (or other sources of Pd or X).

In particular, Tables 3, 4, and 5 group the experimental results in terms of selectivity S and catalytic activity (TOF) for the propylene hydroformylation reaction for different experimental conditions.

In each of these cases, the hydroformylation reaction is carried out under the following conditions:
pressure of the propylene: 4 bar;
pressure of carbon monoxide: 20 bar;
pressure of dihydrogen: 80 bar;
solvent: 2 mL anisole;
imposed temperature: 100° C.

Thus, Tables 3 to 5 show the evolution of the selectivity S and the catalytic activity TOF as a function of the respective proportions (noted “I⁻/Pd”) of the compounds Pd(OAc)₂, PdX₂, PPh₄X, where X corresponds to the anion I−.

In particular, for each of these tables:
the column “Cat.+iodide additive (μmol)” indicates the quantities in μmol (in brackets) of each of the elements Pd(OAc)₂, PdI₂, PPh₄I.;
the column “PCy₃ (μmol)” indicates the amount in μmol of PCy₃;
the column “Pd/L” indicates the molar ratio of the elements Pd to PCy₃ considered.

TABLE 3

| | | Cat. + iodide | PCy₃ | | |
|---|---|---|---|---|---|
| I⁻/Pd | Pd/L | additive (μmol) | (μmol) | TOF | S |
| 0 | 1/2 | Pd(OAc)₂ (10) | 20 | 1.40 | 1.23 |
| 0.5 | | Pd(OAc)₂ (7.5) + PdI₂ | | 10.90 | 1.65 |
| | | (2.5) | | | |
| 1 | | Pd(OAc)₂ (5) + PdI₂ (5) | | 18.83 | 3.69 |
| 1.5 | | Pd(OAc)₂ (2.5) + PdI₂ | | 17.25 | 3.39 |
| | | (7.5) | | | |
| 2 | | PdI₂ (10) | | 16.92 | 3.98 |
| 2.5 | | PdI₂ (10) + PPh₄I (5) | | 12.32 | 4.43 |
| 3 | | PdI₂ (10) + PPh₄I (10) | | 13.29 | 3.24 |

TABLE 4

| | | Cat. + iodide | PCy₃ | | |
|---|---|---|---|---|---|
| I⁻/Pd | Pd/L | additive (μmol) | (μmol) | TOF | S |
| 0 | 1/1 | Pd(OAc)₂ (10) | 10 | 1.93 | 1.69 |
| 0.5 | | Pd(OAc)₂ (7.5) + PdI₂ | | 11.55 | 3.33 |
| | | (2.5) | | | |
| 1 | | Pd(OAc)₂ (5) + PdI₂ (5) | | 9.28 | 3.65 |
| 1.5 | | Pd(OAc)₂ (2.5) + PdI₂ | | 8.93 | 3.54 |
| | | (7.5) | | | |
| 2 | | PdI₂ (10) | | 6.47 | 4.29 |
| 2.5 | | PdI₂ (10) + PPh₄I (5) | | 6.65 | 4.54 |
| 3 | | PdI₂ (10) + PPh₄I (10) | | 9.22 | 2.55 |

TABLE 5

| | | Cat. + iodide | PCy₃ | | |
|---|---|---|---|---|---|
| I⁻/Pd | Pd/L | additive (μmol) | (μmol) | TOF | S |
| 0 | 2/1 | Pd(OAc)₂ (10) | 5 | 0.81 | 1.91 |
| 0.5 | | Pd(OAc)₂ (7.5) + PdI₂ | | 5.77 | 3.10 |
| | | (2.5) | | | |
| 1 | | Pd(OAc)₂ (5) + PdI₂ (5) | | 4.72 | 3.28 |
| 1.5 | | Pd(OAc)₂ (2.5) + PdI₂ | | 3.60 | 3.39 |
| | | (7.5) | | | |
| 2 | | PdI₂ (10) | | 2.64 | 4.78 |
| 2.5 | | PdI₂ (10) + PPh₄I (5) | | 3.23 | 4.79 |
| 3 | | PdI₂ (10) + PPh₄I (10) | | 3.94 | 2.26 |

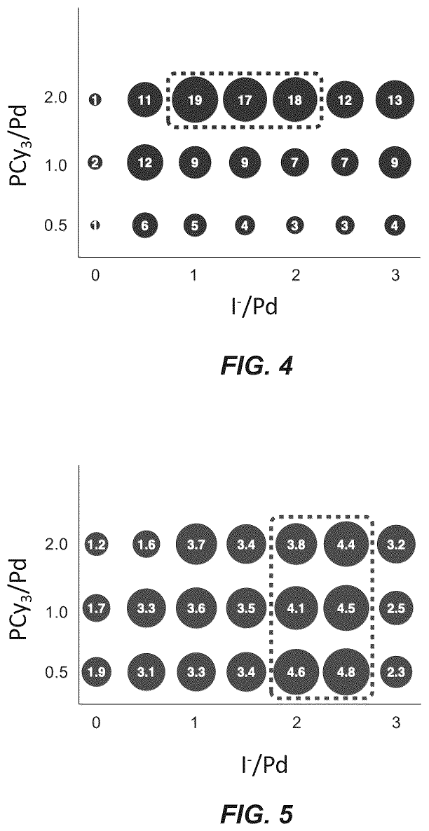

FIGS. 4 and 5 show the results of Tables 3 to 5 in graphical form.

In particular, FIG. 4 graphically represents the evolution of the catalytic activity as a function of the L/Pd ratio (vertical axis) and the I⁻/Pd ratio (horizontal axis), while FIG. 5 graphically represents the evolution of the selectivity S as a function of the L/Pd ratio (vertical axis) and the I⁻/Pd ratio (horizontal axis).

Thus, in accordance with the results illustrated in FIG. 5, a molar ratio of the halogen element X to palladium of between 1 and 3, advantageously between 2 and 2.5, makes it possible to achieve a selectivity S of more than 3 or even more than 4.

Equivalently, the results illustrated in FIG. 5, with a molar ratio of phosphine P(R)(R′)(R″) to palladium of between 1 and 4, advantageously between 2 and 3, makes it possible to achieve a catalytic activity greater than 7, or even greater than 15.

The present disclosure shall not be limited to the sole consideration of the aforementioned ligands.

In particular, the skilled in the art can further consider the ligand or group of ligands listed in Table 6, Table 7, Table 8, and Table 9 and wherein both TOF and the selectivity associated with of each ligand or group of ligands is indicated.

TABLE 6

| | | Selectivity |
|---|---|---|
| Ligand | TOF/h⁻¹ | (S) |
| Davephos | 13.0 | 1.47 |
| Di(1-adamantyl)-1- | 10.4 | 1.75 |
| piperidinylphenylphosphine | | |
| P(tBu)2(4-Me2NPh) | 7.7 | 2.21 |
| CyJonhphos | 7.4 | 2.18 |
| 2-(Dicyclohexylphosphino)- | 7.0 | 2.59 |
| 1-phenyl-1H-pyrrole | | |
| P(OPh)3 | 6.9 | 1.61 |
| PCy2Ph | 6.7 | 4.40 |
| Mephos | 5.0 | 1.71 |
| PPh2OMe | 4.8 | 1.24 |
| PPh2(2-Py) | 4.7 | 1.59 |
| Cybippyphos | 4.3 | 2.29 |
| CTC-Q-PHOS | 4.0 | 1.78 |

TABLE 7

| | | Selectivity |
|---|---|---|
| Ligand | TOF/h⁻¹ | (S) |
| PPh2(t-Bu) | 3.7 | 2.27 |
| PCy2(4-Me2NPh) | 3.0 | 4.98 |
| PEt3 | 3.0 | 1.66 |
| P(OCH2CF3)3 | 2.9 | 1.91 |
| Bippyphos | 2.9 | 1.32 |
| Cy-vBRIDP | 2.5 | 2.08 |
| Trippyphos | 2.4 | 1.27 |
| CPhos | 2.2 | 1.18 |
| Me4tButylXphos | 2.0 | 2.06 |
| 2-[Bis(3,5-di-tert-butyl-4- | 2.0 | 2.29 |
| methoxyphenyl)phosphino]benzaldehyde | | |
| P(OEt)3 | 1.8 | 2.72 |
| P(t-Bu)3 | 1.7 | 1.93 |

TABLE 8

| | | Selectivity |
|---|---|---|
| Ligand | TOF/h⁻¹ | (S) |
| Sphos | 1.7 | 1.26 |
| Johnphos | 1.6 | 0.93 |
| 5-[Di(1-adamantyl)phosphino]-1′,3′,5′- | 1.6 | 1.08 |
| triphenyl-1′H-[1,4′]bipyrazole | | |
| 2-(Di-tert-butylphosphino)-1-phenylindole | 1.5 | 1.28 |
| P(4-ClPh)3 | 1.4 | 1.17 |
| PPh2(2OHPh) | 1.4 | 2.16 |
| P(OSi(Me3))3 | 1.3 | 1.27 |
| ((2,4,6-Tri-isopropyl)phenyl)di- | 1.3 | 1.23 |
| cyclohexylphosphine | | |
| P(4-MePh)3 | 1.2 | 1.00 |
| PPh2(p-tolylPh) | 1.2 | 1.29 |
| vBRIDP | 1.2 | 1.10 |
| P((CH2)3OH)3 | 1.1 | 2.01 |

TABLE 9

| | | Selectivity |
|---|---|---|
| Ligand | TOF/h⁻¹ | (S) |
| P(OMe)3 | 1.0 | 1.67 |
| PPh2(OEt) | 1.0 | 1.10 |
| PPh2Bn | 1.0 | 1.73 |
| P(O(t-Bu))3 | 0.9 | 1.24 |
| PPh2H | 0.9 | 2.79 |
| TrixiePhos | 0.8 | 1.61 |
| Diphenyl(2-methoxyphenyl)phosphine | 0.8 | 2.58 |
| P(pyrrolidinyl)3 | 0.7 | 5.18 |
| 2-(Di-tert-butylphosphino)-1-(2- | 0.7 | 1.53 |
| methoxyphenyl)-1H-pyrrole | | |
| 4-(diphenylphosphino)benzoic acid | 0.7 | 1.93 |
| P(3,5-Me2Ph)3 | 0.7 | 1.39 |
| R-MOP | 0.6 | 1.34 |
| (3aR,8aR)-(−)-(2,2-Dimethyl-4,4,8,8- | | |
| tetraphenyl-tetrahydro-[1,3]dioxolo[4,5- | | |
| e][1,3,2]dioxaphosphepin-6-yl)dimethylamine | 0.6 | 2.65 |
| PPh2Cy | 0.3 | 2.91 |

It is also admitted that ligands or a group of ligands listed in Table 6, Table 7, Table 8, and Table 9 that are associated with a selectivity below 1 can be considered for the implementation of hydroformylation of propylene with carbon monoxide and hydrogen to selectively form n-butanal with respect to iso-butanal.

Of course, the present disclosure is not limited to the embodiments described and alternative embodiments may be made without departing from the scope of the invention as defined by the claims.

### REFERENCES

[1] Ning, Y. et al, “Transition Metal-Catalyzed Branch-Selective Hydroformylation of Olefins in Organic Synthesis” Green Synth. Catal. 2021, S2666554921000399;
[2] U.S. Pat. No. 8,710,275B2;
[3] Iu, L. et al, “High Iso Aldehyde Selectivity in the Hydroformylation of Short—Chain Alkenes,” Angew. Chem. Int. Ed. 2019, 58 (7), 2120-2124.

1. A method for the hydroformylation of propylene with carbon monoxide and hydrogen to selectively form iso-butanal with respect to n-butanal, the method comprising combining a hydroformylation catalyst with a phosphine, the hydroformylation catalyst comprising a palladium catalyst comprising PdX₂, where X is a halogen element selected from iodine I and bromine Br, and the phosphine is of the form P(R)(R′)(R″), where R, R′ and R″ each comprise an alkyl group or an aryl group or an alkoxy group or an amino group.

2. The method of claim 1, wherein the phosphine is selected from among the following group: P(Me)₃, P(Et)₃, P(n-Bu)₃, P(n-Hex)₃, P(i-Bu)₃, P(i-Pr)₃, P(Cy)₃, P(Cyp)₃, P(t-Bu)₃, P(Bn)₃, P(Cy)₂(t-Bu), P(Cy)(t-Bu)₂, P(Me)(t-Bu)₂, P(i-Pr)₂(t-Bu), P(1-Ad)₂(n-Bu), P(Ph)(t-Bu)₂.

3. The method of claim 1, wherein a molar ratio of the halogen element X to palladium is in a range extending from 1 to 3.

4. The method of claim 3, wherein the halogen element X is iodine I.

5. The method of claim 3, wherein the halogen element X is bromine Br.

6. The method of claim 1, wherein a molar ratio of phosphine P(R)(R′)(R″) to palladium is in a range extending from 1 to 4.

7. The method of claim 1, wherein the phosphine comprises P(Cy)₃.

8. The method of claim 1, wherein the palladium catalyst comprises an additional palladium component.

9. The method of claim 1, wherein the method is carried out in a non-aqueous solvent comprising at least one substance selected from among the following group: anisole, toluene, 1,4-dioxane, dimethylacetamide, p-xylene, decahydronaphthalene, DMF, DMSO, α,α,α-trifluorotoluene, hexafluorobenzene, 1,2-dichlorobenzene, methyl benzoate.

10. The method of claim 1, wherein the method is carried out at a temperature in a range extending from 70° C. to 200° C.

11. The method of claim 1, wherein R, R′ and R″ are chosen so that the selectivity S of the hydroformylation of propylene with carbon monoxide and hydrogen is above 2.

12. The method of claim 3, wherein the molar ratio of the halogen element X to palladium is in a range extending from 2 to 2.5.

13. The method of claim 12, wherein the halogen element X is iodine I.

14. The method of claim 12, wherein the halogen element X is bromine Br.

15. The method of claim 6, wherein the molar ratio of phosphine P(R)(R′)(R″) to palladium is in a range extending from 2 to 3.

16. The method of claim 6, wherein the phosphine comprises P(Cy)₃.

17. The method of claim 8, wherein the additional palladium component comprises Pd(OAc)₂.

18. The method of claim 10, wherein the method is carried out at a temperature in a range extending from 80° C. to 120° C.

19. The method of claim 11, wherein R, R′ and R″ are chosen so that the selectivity S of the hydroformylation of propylene with carbon monoxide and hydrogen is above 3.
